(19) United States
(12) Patent Application Publication
(10) Pub. No.: US 2007/0255122 A1
(43) Pub. Date: Nov. 1, 2007

(54) Device and Method for Measuring Physiological Parameters

(75) Inventors: Alexander Vol, Rehovot (IL); Orna Gribova, Rehovot (IL)

Correspondence Address: BROWDY AND NEIMARK, P.L.L.C.;624 NINTH STREET, NW, SUITE 300, WASHINGTON, DC, 20001-5303, US

(73) Assignee: G.R. Enlightenment Ltd., Tel Aviv (IL)

(21) Appl. No.: 11/661,422

(22) PCT Filed: Aug. 30, 2005
(86) PCT No.: PCT/IL05/00926

§ 371 Date: Feb. 28, 2007

(30) Foreign Application Priority Data
Aug. 30, 2004 (IL) 163796

(51) Int. Cl. A61B 5/0205

(52) U.S. Cl. 600/301

(57) ABSTRACT

Device and method for measuring physiological parameters of a biological being comprising: at least two spaced apart electrodes at least one of which is in contact with-the being for providing a bio-potential measurement including a low frequency AC voltage and/or a DC voltage wherein one of the electrodes is a reference electrode providing a reference for the DC voltage, the low frequency AC voltage and/or DC voltage measurement used to determine the physiological parameters. The device can be built in many forms (e.g. a wrist watch, torso strap, grip, etc); can measure physiological parameters including those related to diabetes (BGL), cardiovascular, organ, tissue, brain and neural function, local and limb metabolic condition, pharmacokinetic, pharmaco-dynamics and psychological conditions, temperature, or combination thereof and their trends. The device can include an automatic alarm system for warning a patient of an out of tolerance condition.

### FIELD OF INVENTION

The present invention relates to a device and method particularly those that measure physiological parameters of a biological being.

### BACKGROUND OF THE INVENTION

There are many devices available for directly measuring or estimating a biological being's vital physiological parameters (e.g. blood glucose level, cardiovascular functioning, etc.) and the monitoring those parameters. Many of these techniques are non-invasive or essentially non-invasive, which is usually simpler and more comfortable for the biological being.

For example, cardiovascular monitoring and glucose monitoring devices have been developed using a variety of techniques. These techniques typically are based on correlation between a cardiovascular state and heart electrical activity (e.g. as measured by an ECG sensor).

Electrocardiography and/or Echocardiography are also used to monitor certain health parameters and uses electrical, acoustic sensors and optical pulse wave detectors (e.g. as disclosed in U.S. Pat. No. 6,921,367, which describes estimating hemoglobin, glucose and oxygen concentrations in the blood).

U.S. Pat. No. 6,920,348 discloses a system and method for determining metabolic factors using electrocardiogram measurements from a person's Wilson points. A first derivative of an electrocardiogram measurement is calculated. A ratio is calculated of the absolute value of the positive spike of the first derivative to the sum of the absolute values of the positive and negative spikes. In some embodiments, the ratio is multiplied by a constant to determine metabolic factors. Further operations may be performed on the ratio to determine other metabolic factors. In some embodiments, a garment is provided for easily locating the Wilson points.

U.S. Pat. No. 6,925,324 discloses a medical device and method for analyzing physiological and health data and representing the most significant parameters. Low, intermediate and high-resolution scales can exchange information between each other. The low-resolution scale represents a small number of primary elements such as intervals between the heart beats, duration of electrocardiographic PQ, QRS, and QT-intervals, amplitudes of P-, Q-, R-, S-, and T-waves. This real-time analysis is implemented in a portable device that requires minimum computational resources. In the intermediate-resolution scale, serial changes in each of the elements can be determined using a mathematical decomposition into series of basis functions and their coefficients. This scale can be implemented using a specialized processor or a computer organizer. At the high-resolution scale, combined serial changes in all primary elements can be determined to provide complete information about the dynamics of the signal. This scale can be implemented using a powerful processor, a network of computers or the Internet. The system can be used self-evaluation, emergency or routine ECG analysis, or continuous event, stress-test or bed-side monitoring.

U.S. Pat. No. 5,741,211 discloses a system and method for sensing and providing an indication of one or more diabetes-related blood constituents (e.g. insulin or glucose). The system is based on an ECG sensor which can be an external wearable device or an implantable one.

U.S. Pat. No. 6,022,321 describes an apparatus for detecting pulse waves and motion intensity comprising photo-coupler type photo-sensors which are attached to a biological being and provide body motion information superimposed on blood pulse signals which are analyzed by a Fourier transformation.

U.S. Pat. No. 6,334,850 discloses an optical type pulse wave device suitable for detecting a pulse waveform according to blood flow through an artery or blood vessels around the artery.

U.S. Pat. No. 6,645,142 describes a glucose monitoring instrument having network-based communication features which provide a link between patient and practitioner.

U.S. Pat. No. 6,704,588 provides an apparatus for determining a diagnostic glucose level using collimated light at a selected wavelength which computes glucose concentration based on measured polarization and the optical path length.

U.S. Pat. No. 6,675,030 discloses an individualized modeling equation for predicting a patient's blood glucose values generated as a function of non-invasive spectral scans of a body part and an analysis of blood samples from the patient, and is stored on a central computer.

U.S. Pat. No. 6,723,048 describes an apparatus for non-invasive detection and quantifying of analytes, such as blood glucose, employing an amplifier that uses high-gauss permanent magnets to permit an RF signal to be transmitted through the sample. The concentration of the analyte can be determined from the magnitude of the reduction in the amplitude of the radio-frequency (RF) signal at a characteristic frequency.

U.S. Pat. No. 6,728,560 describes an optical tissue glucose device provides a measurement of the glucose level in mucous. The instrument may comprise a radiation source capable of directing radiation to a portion of the exterior or interior surface of a patient. That surface may be a mucosal area such as the gums and other mucosal areas, the eyeballs and surrounding areas such as the eyelids and, preferably, the skin.

In addition, numerous articles and experiments have been published relating to measuring and analyzing physiological health parameters.

### SUMMARY OF THE INVENTION

The present invention relates to a device and method for measuring, recording and analyzing the electrical, magnetic, bio-mechanical, acoustic, metabolic activity of a biological being or parts thereof. The present device and method can be used to measure physiological parameters including blood glucose level, insulin sensitivity, nervous system state, cardiovascular function (including heart rate, blood viscosity, blood pressure, pulse wave area and pulse spectrum), other organ function (including the brain), tissue function, metabolic condition (including cancer diagnostics), and so on.

The term biological being is used herein below and in the claims in its broadest sense and can include people, animals or plants—healthy or non-healthy. These beings need not be voluntary “patients”, for example in the case of terrorists, criminals, etc as will be discussed below. As the more common applications relate to people, and more particularly “patients”, the terms may be used interchangeably herein, without implying limitation of the scope of the present invention.

By one aspect thereof, the present invention provides a device for measuring physiological parameters of a biological being comprising: at least two spaced apart electrodes at least one of which is in contact with the biological being for providing a bio-potential measurement including a low frequency AC voltage and/or a DC voltage in which one of the at least two electrodes is a reference electrode providing a reference for the DC voltage, wherein the low frequency AC voltage and/or DC voltage of the bio-potential measurement is used to determine the physiological parameters.

By one aspect thereof, the present invention provides a method for measuring physiological parameters of a biological being comprising: (a) providing a device according to any of the previous claims; (b) contacting the device with a biological being, (c) measuring at least a DC voltage and/or a low frequency AC voltage of the biological being.

The combination of electrodes constituting the basic building block (BB) of the device is constituted by: two spaced apart electrodes at least one of which is in contact with the biological being for providing a bio-potential measurement including a low frequency AC voltage and/or a DC voltage in which one of the two electrodes is a reference electrode providing a reference for the DC voltage.

Additional sensors may be added to the basic building block or BB whereby the device may be used to either measure additional physiological parameters or allow the device to be used in more complicated settings. For example, the device may include a motion sensor whereby the biological being may be physically active while using the device and such activity may be taken into account during analysis of the measurements.

The term low frequency AC voltage refers herein to AC voltages generally below about 0.7 Hz (whereas present ECG, EMG and EEG devices use high frequency AC voltage i.e. typically above 0.7 Hz).

The device can be adapted to be a comfortable, non-invasive, and inexpensive measuring, analysis and monitoring device, which may comprise or be used with a wireless multi-electrode system, and which can continuously detect physiological parameters and provide rapid output.

The biological beings may be described as a multi-dimensional space of entropy and interdependent parameters. In a first approximation it can be modeled as multi-parametric relaxation oscillator. Such an approach has enabled development of the present invention, which is a multi-parametric measurement system that allows multi-diagnostics with a number of specific applications.

Such an approach has enabled development of the present invention, which, according to particular embodiments is a dynamic, multi-parametric measurement device that allows simultaneous multi-diagnostics with a number of specific applications.

The device uses a combination of electrical sensors to obtain a DC voltage measurement and low-frequency AC measurements in addition to standard “high frequency” measurements (above 0.7 Hz) of the bio-potential as commonly measured by ECGs, EMGs and EEGs together with passive sensors (i.e. they do not input energy into the biological being).

By particular embodiments, the device further provides, singularly or in combination, a wireless ECG, EMG, EEG and brain hemisphere electrical activity sensor.

Different combinations of the developed sensors facilitate real time diagnosis of different illnesses including cancers, because illness and cancer are essentially a deviation in the local metabolism, and real time observation and measurement of pharmaco-kinetics and pharmaco-dynamics. It may be further used in pharmacological industry for medication development and individual adjustment existing treatment protocols. It may be used also for sport training, refining diet program, lie detector machines, chakra diagnostics, pregnancy and other types of tracking of physiology state diagnostics.

In addition to using combination of electrical sensors to obtain a DC voltage measurement and/or low-frequency AC measurement, the invention may further comprise standard “high frequency” measurements of the bio-potential as commonly measured by ECGs, EMGs and EEGs, together with passive physical sensors including accelerometer(s), mechanical sensors and acoustic and temperature sensors that measure and allow recording or electrical and acoustic activity, motion and shape and rate of pulse wave propagation.

According to particular embodiments, the device and method are used on a developed organism using thermodynamic theory which allows estimation of the blood glucose level, insulin sensitivity, nervous system and cardiovascular state including blood pressure and blood viscosity, local basic metabolism of inner organs and limbs and other parameters of a biological body's physiological state. Different combinations of the sensors facilitate real time diagnosis of different illness including cancers, because any illness and cancers are essentially is a deviation in the local metabolism. The invention also allows real time observation and measurement of pharmacokinetics and pharmaco-dynamics.

It can be used as a blood glucose level monitor, limb metabolism monitor, wireless ECG device, pharmaco-dynamics tracking system, nervous activity sympathetic/parasympathetic index estimator, lie detector, local metabolism disorder diagnostic device and so on.

It is important to note that at least certain embodiments of the device may be used as a biofeedback systems in order to help a physician (or the patient himself), in real time, to choose or correct a health protocol or treatment and for medication development and treatment protocols including biofeedback for determining medication efficacy. It may be used also for sport training, refining diet program, lie detector machines, pregnancy and other types of tracking of physiology state diagnostics.

In particular embodiments, the electrodes provide a measurement of DC and AC voltages and time propagation of the electrical wave between any two electrodes. A reference electrode for providing a reference for the DC voltage measurement may be, for example, a saturated AgCl electrode.

These electrodes may be positioned along a limb (e.g. at a wrist or ankle) at a cross-section of the limb, or along the direction of blood flow, allowing an estimation of the hand/foot metabolic state at different blood glucose levels. The device could alternatively/further comprise an array of electrodes (e.g., a multi-electrode pad network), which can be placed on any part of the biological being and provide measurement of AC and DC voltages and time propagation of the electrical wave along of any direction of such electrode network.

The above-mentioned accelerometer can provides a measurement of body movement and detect tremors, for example that may that take place under hypoglycemic conditions. This accelerometer may be connected to a microprocessor that allows an estimation of the complete motion accuracy and coordination and metabolic state of a patient under different psycho-immune conditions and at different blood glucose levels.

Note: acoustic and accelerometer sensors may have different spectral characteristics and so should typically be used with different contact and placement at the body parts. For example, a microphone may be placed on the body using air or another gas as a working conductive medium. This helps prevent high frequency oscillations that take place in solid and liquid media. On the other hand, accelerometers preferably use a liquid or semi-liquid contact with body surface. In this case all high frequency oscillations up to about 300 kHz may be measured and recorded by a transducer that allows observation of longitudinal and cross sectional waves, in the bones or other matter, which enables diagnosis and observation of joint and bone function, damage, wear, etc.

According to further embodiments of the present invention, the device/method may include a thermal regulation and disease condition and comprises at least two biocompatible temperature sensors, for example thermo-couples or thermo-resistors, providing a measurement of skin and surrounding temperatures. The resultant temperature measurements allow an estimation of the thermo-regulation status under different external or internal conditions (e.g. disease) that affects blood flow, metabolism and glucose and insulin consumption.

The present invention may further include a programmable microprocessor, which allows personal calibration, for example, of the device as use as a glucose monitor. The programmable microprocessor allows necessary parameters to be input during periodic clinical examination of a diabetic patient. Such clinical examination may include an oral glucose tolerance test (OGTT). Measurement of the postprandial increase of blood glucose level may be used also for calibration. Calibration generally includes routine laboratory analyses of blood glucose levels and their correlation with physiological parameters.

In particular embodiments, the device may comprise a perspiration indicator and perspiration acidity combined sensor having at least two biocompatible electrodes made from different conductive materials, the perspiration constituting a conductive electrolyte so as to form a galvanic electricity source. The voltage and current depends on existence and acidity of the perspiration. Such an element does not need an external source of electricity thus increasing the life and reliability of the system.

The device can be actualized in different forms, for example:
1. A wrist-watch or anklet comprising a pair of pulse wave sensors, which provide data to produce a shape and time of propagation of the pulse wave between the sensors for use in determining limb metabolism, cardiovascular condition, nervous system measurement device; or a glucose monitoring device.
2. Belts or pads having sensors attached to the body for measuring local metabolism, brain activity, pharmacokinetics or pharmaco-dynamics; or for use in a lie detector machine or cancer diagnostics.
3. A wireless clothing article where all signals continuously in real time transmit signals (e.g. infra-red, ultra-sound, etc.) to a central receptor station (processor) allowing a person free movement for participating in sports or other daily activity.
4. A grip, rod, housing, surface, for instance to be touched, grasped and so on.
5. An invasive type device.
6. A combination of the above-mentioned forms.

The following theoretical thermodynamic analysis is the basis for all of the embodiments of the present device and method for measuring the abovementioned physiological parameters. It is important to mention that although mainly diagnostic embodiments are discussed, the device may be used as a biofeedback system, for example, to help a physician, or the patient himself, in real time, to choose or correct a health protocol or treatment.
1) O₂ & CO₂ transport rate from capillaries into interstitial fluid is diffusion controlled (concentration gradient controlled, i.e. by the difference between the partial pressure of the gases in the interstitial fluid and arterial/venous capillaries)
2) Energy consumption and CO₂ production is essentially constant in a biological being's rest condition and it corresponds to the “basic metabolism”.
3) Increased metabolic activity may be caused by physical activity, the environment including thermal control by the body or by disease. It leads to increased formation of CO₂ and probably lactic acid. The increased CO₂ concentration affects the equilibrium reaction CO₂+H₂O═HCO₃⁻¹+H⁺ thereby affecting the electrolyte concentration (e.g. NaHCO₃, KHCO₃, CaCO₃).
4) Thus, an increase in metabolic intensity (e.g. due to disease) affects electrolyte concentration in the cells and interstitial fluid and so the liquid acidity (lower or higher pH), resulting in a change in redox potential. The metabolic intensity caused by disease, metabolic problems, etc, can be isolated from other causes by the application of appropriate algorithms.
5) For each 0.1 pH change there is a DC voltage change of approximately 6 m VDC for (i.e. a 0.1 pH increase results in a 6 m VDC increase).
6) Thus, gas and metabolite transport is accompanied by a DC potential difference.
7) Diseased cells are accompanied by increased metabolic activity and thus increased CO₂ concentration, and, as understood from the above, an increased DC voltage. Thus, a DC voltage can be used to indicate an unhealthy situation. However, that increased activity may merely be physical activity so that one must first correlate the DC change with physical activity to get a baseline.

The theory takes into account the principally different dynamic characteristics of glucose transport, and other metabolite transport, from blood vessel capillary walls to/from interstitial fluid. Note, diffusion has a linear rate dependence on concentration gradient and area of capillary walls to/from interstitial fluid and transport rate through cellular membranes depends on insulin concentration, receptor state, and carrier concentration and may be energy dependent, or non-dependent. The interstitial fluid partially compensates for local and/or temporal rate differences of the linear and non-linear parts of the metabolic transport and analysis of this dynamics allows estimation of the above listed and other important physiological parameters.

When a body's physiological parameters are in the normal range, the quality of physiological control is maximal and rate return to homeostasis is maximal also. When one or more of physiological parameters are out of the normal tolerance range, the quality of the body control is decreased and oscillations that are typical of such a non-tolerance range condition are observed.

Such a decrease in the quality of a body's control is understandable, because metabolite transport is a combination of linear and non-linear processes. For example, an athlete may use aerobic and anaerobic respiration despite the fact that anaerobic respiration is much less efficient. In this case muscles and other tissues accumulate products of fermentation like lactic acid and other acids in interstitial fluid. Similar processes take place under intolerance of glucose or a disease condition.

Most metabolite transport through cellular membranes may be described by the well known Michaelis-Menten equation. It relates to non-linear processes that act in series with, in the present case, linear transport through blood and lymph capillaries. It is known that the restoration rate back to equilibrium is faster when the physiological parameters are within the tolerance range.

Deviation outside of normal physiological tolerance ranges causes a decrease in the quality of body control processes and is accompanied by over-regulation (oscillations). Provided by the present invention is dynamic on-line tracking of physiological changes allowing discrimination of different types of parameters deviations. Using the device and method with personal calibration, allows an individual mathematical model to be built for the determination of the blood glucose level, nervous system and cardiovascular state, pharmaco-kinetics and pharmaco-dynamics, etc.

An interesting example of such an approach results from a comparison of healthy cells and cancer cells. The more primitive metabolism of the cancer cells leads to increase in the Gibbs energy of these cells relative to the health cells, which are close to a normal homeostasis condition (i.e. not in a range of particularly low or particularly high metabolism). The polymorphic characteristics of cancer cells may be estimated as a differential change in the Gibbs energy divided by the Plank constant.

Regardless, the Gibbs energy is lower in cancer cells under the both too low or too high metabolic conditions. It is one reason why people reaching the end of the reproductive life-period have a higher probability of breast, prostate and uterus cancers.

It is very important to note also that the stability of cancer cells is more limited by an increase in entropy than healthy cells. Therefore particularly those (cancer) cells are more sensitive to hyperthermia, which is used today as an effective cancer therapy. However, hyperthermia cannot be effective under either too poor or too high metabolic conditions (this will be understood better with reference to FIG. 6, described below). This treatment can work if the patient is close to the normal homeostasis. For example, for women close to menopause it is important in addition to the hyperthermia to give a hormonal treatment which will normalize the blood circulation in the reproductive organs.

Another example supporting the theory used in the present invention is brain function during coordinated movement. It is well know that symmetric movements are easier in performance then non-symmetric ones.

The quality of the movement coordination is very important parameter of the nervous system. Strong emotional or physical stress decreases the quality of nervous control. Therefore the coordination itself in combination with other measurable physiological parameters may be used for the measurement of the psycho-immuno-physiological state. Examples where this measurement may be used is in checking people working in positions of great responsibility like airplanes, nuclear-power stations, etc., or as part of a regular health screening or to detect possible terrorists, criminals etc. who likely tend to exhibit emotional or physical stress, which may be measurable by the device of the present invention.

### DETAILED DESCRIPTION OF THE DRAWINGS

The invention may be more clearly understood upon reading of the following detailed description of non-limiting exemplary embodiments thereof, with reference to the following drawings, in which:

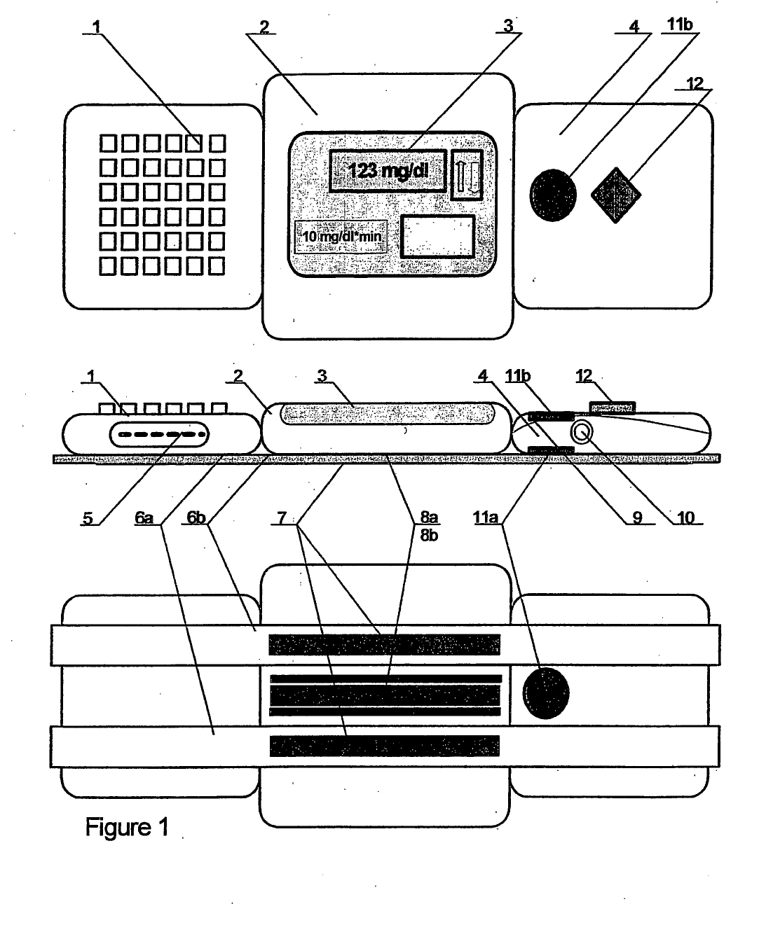

FIG. 1 shows schematic depictions of the glucose monitoring device, viewed from the top, in cross-section and from the bottom, respectively, according to an embodiment of the present invention;

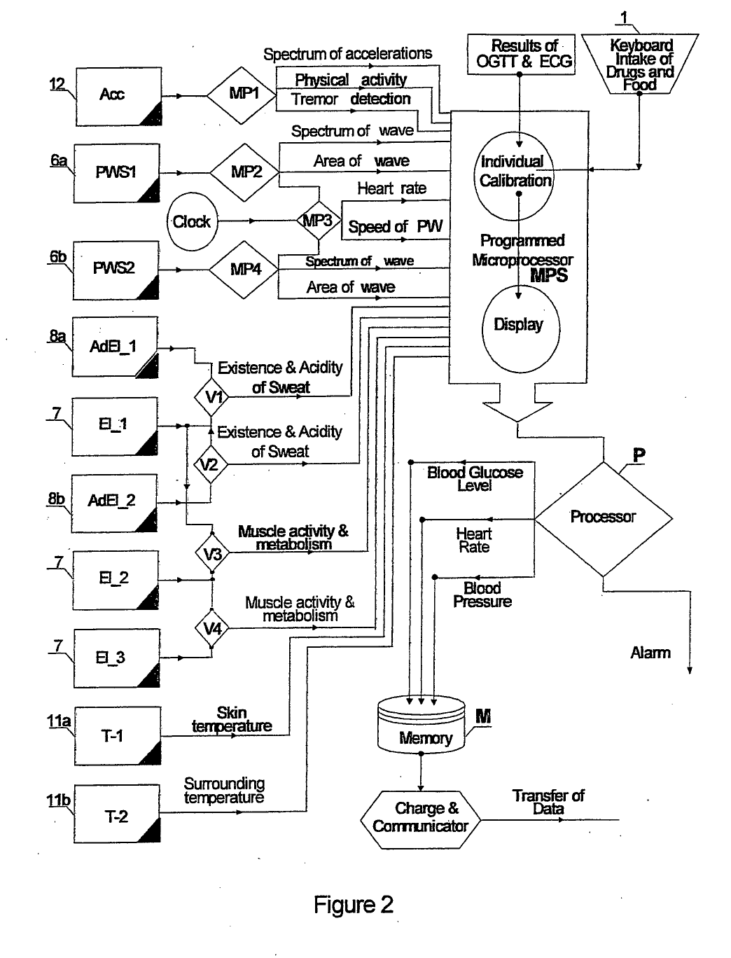

FIG. 2 is a block-diagram showing the operating logic of the glucose monitoring device of FIG. 1;

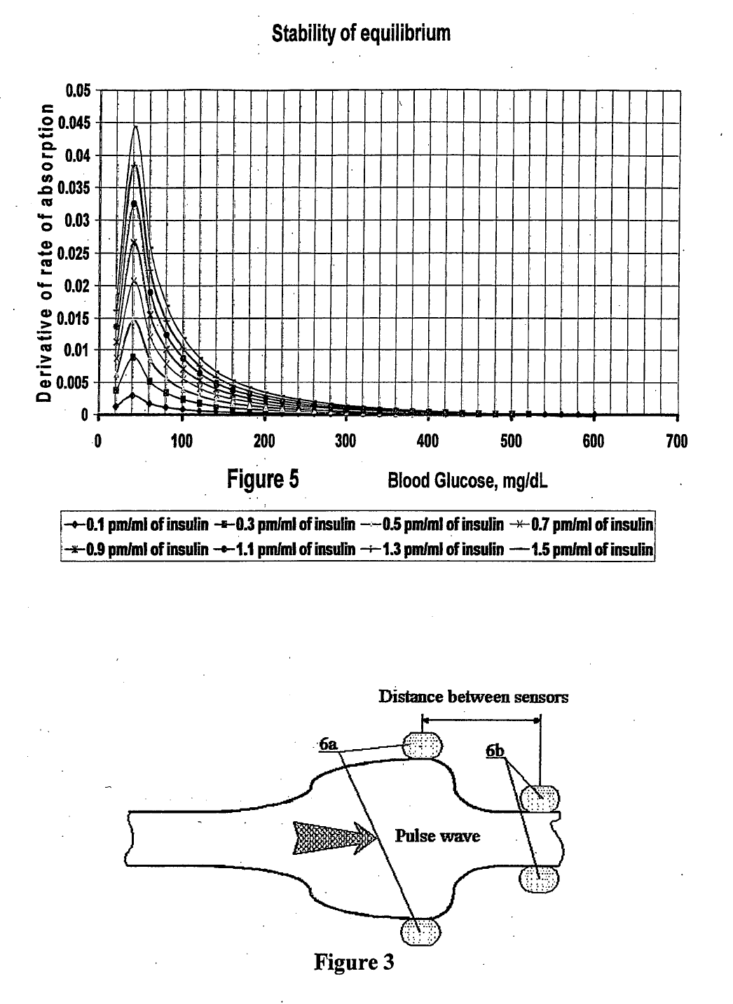

FIG. 3 is a diagram illustrating the measurement principles of a pulse wave and its propagation rate used in the present invention;

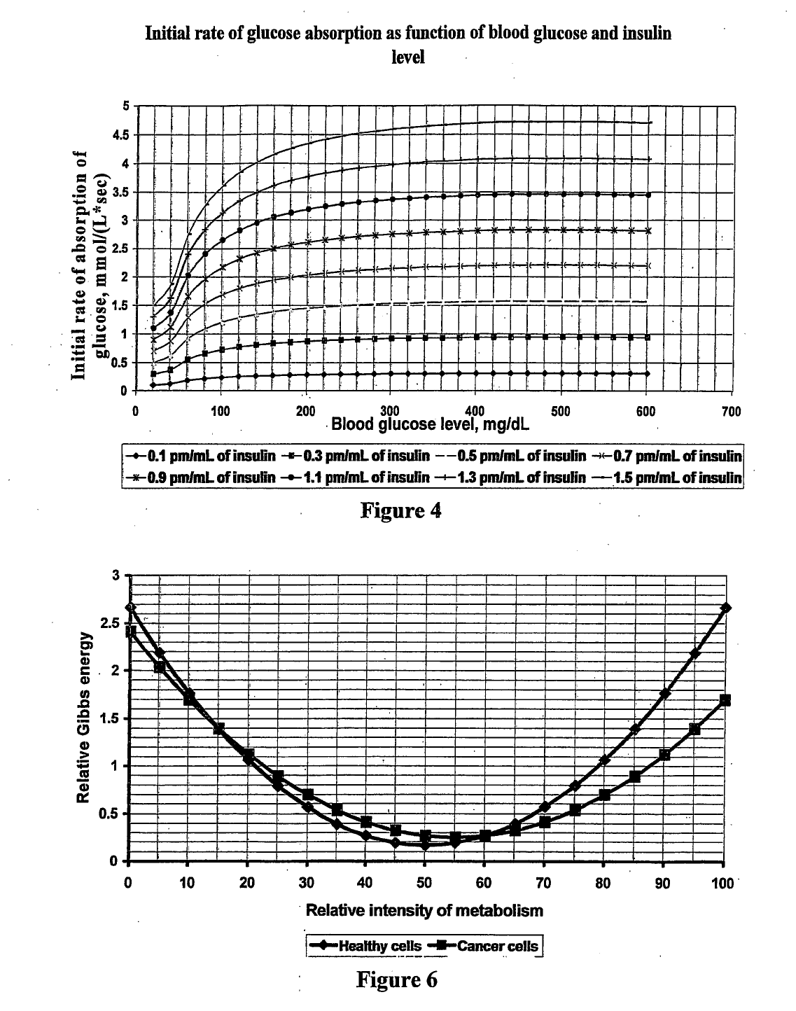

FIG. 4 is a graph showing the rate of glucose absorption as function of blood glucose and insulin levels according to a theoretical estimation;

FIG. 5 is a derivative rate of glucose absorption which reflects restoration rate of the metabolic equilibrium, in other words, system stability;

FIG. 6 shows the Gibbs energy of healthy and cancer cells;

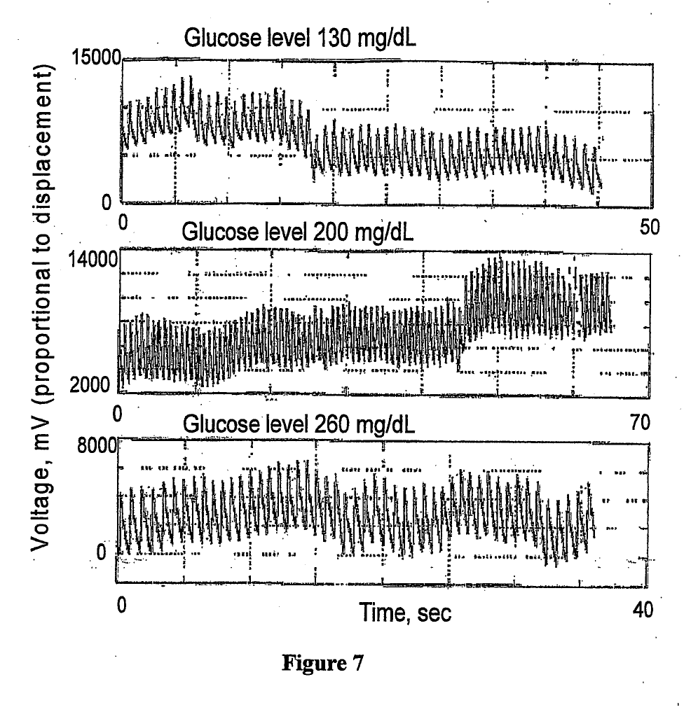

FIG. 7 shows graphs of raw experimental data from pulse wave sensors at three blood glucose levels;

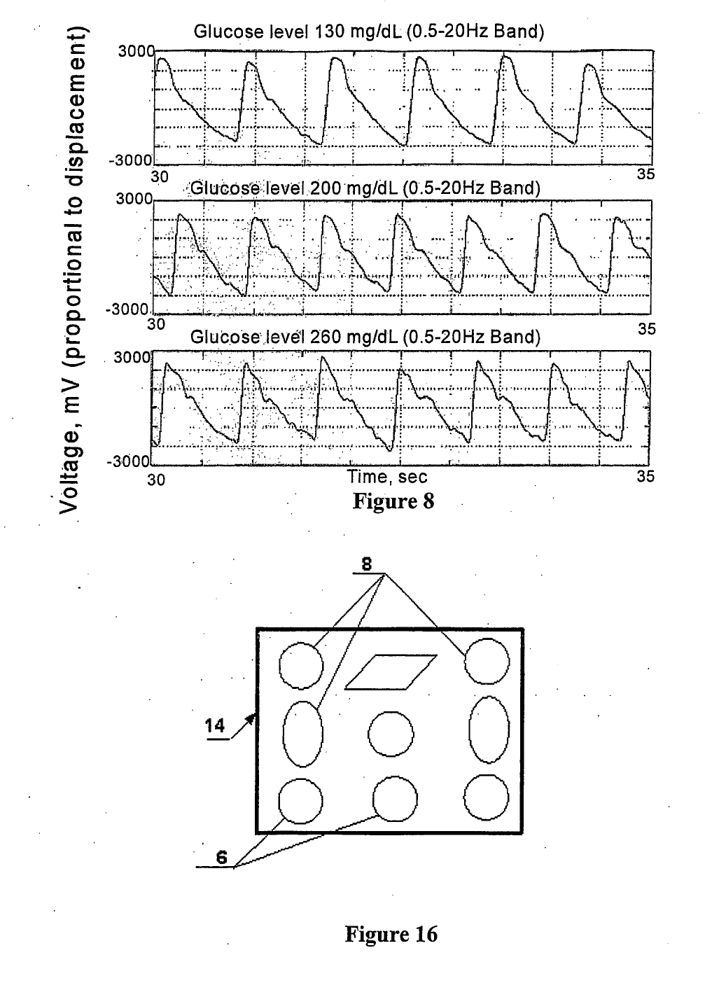

FIG. 8 shows graphs displaying the experimental data of FIG. 6, after data filtering;

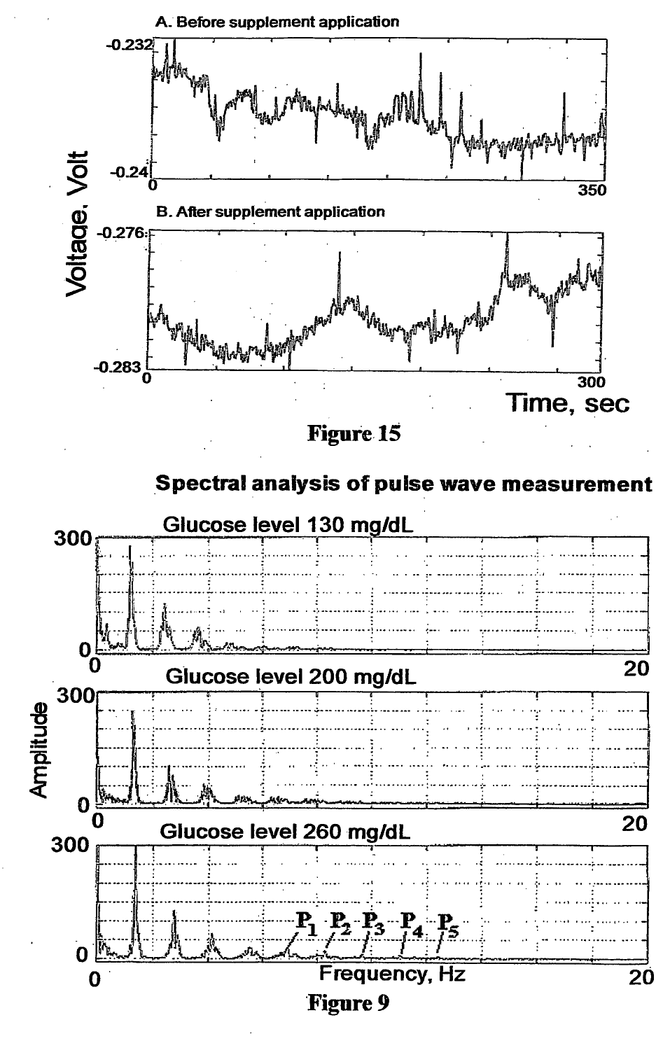

FIG. 9 shows the result of Fourier analysis on the data displayed in FIG. 7;.

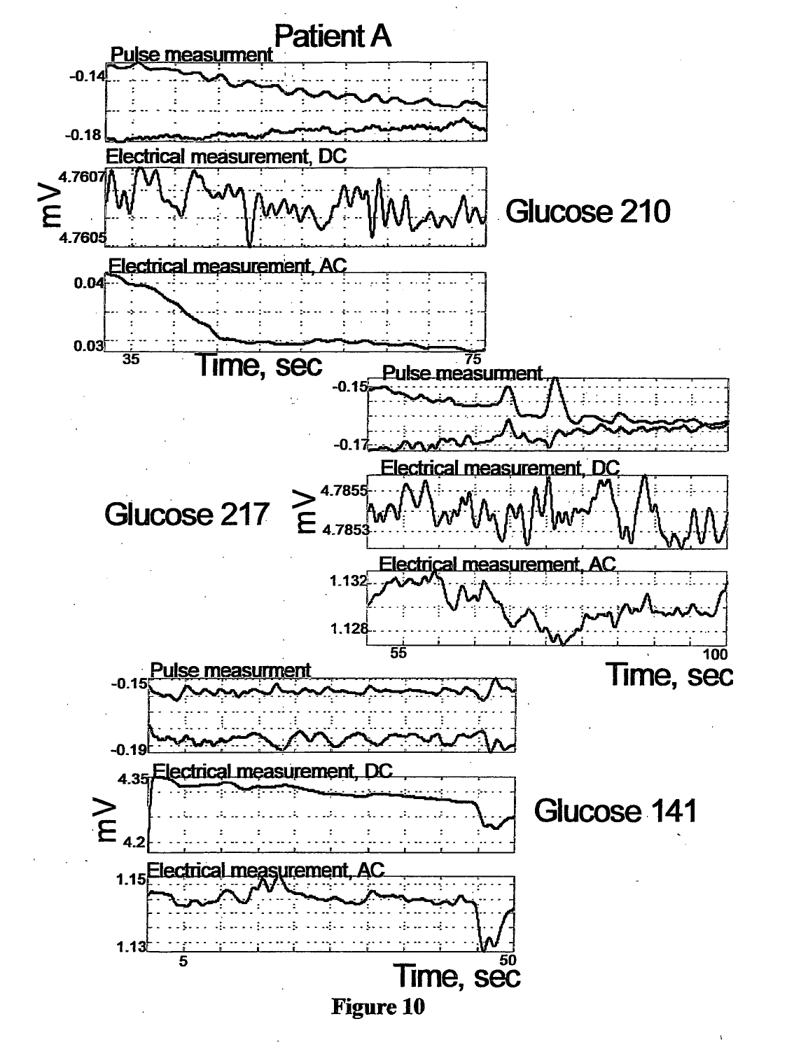

FIG. 10 is a graph showing experimental data generated by the present invention for a diabetic patient at three blood glucose levels;

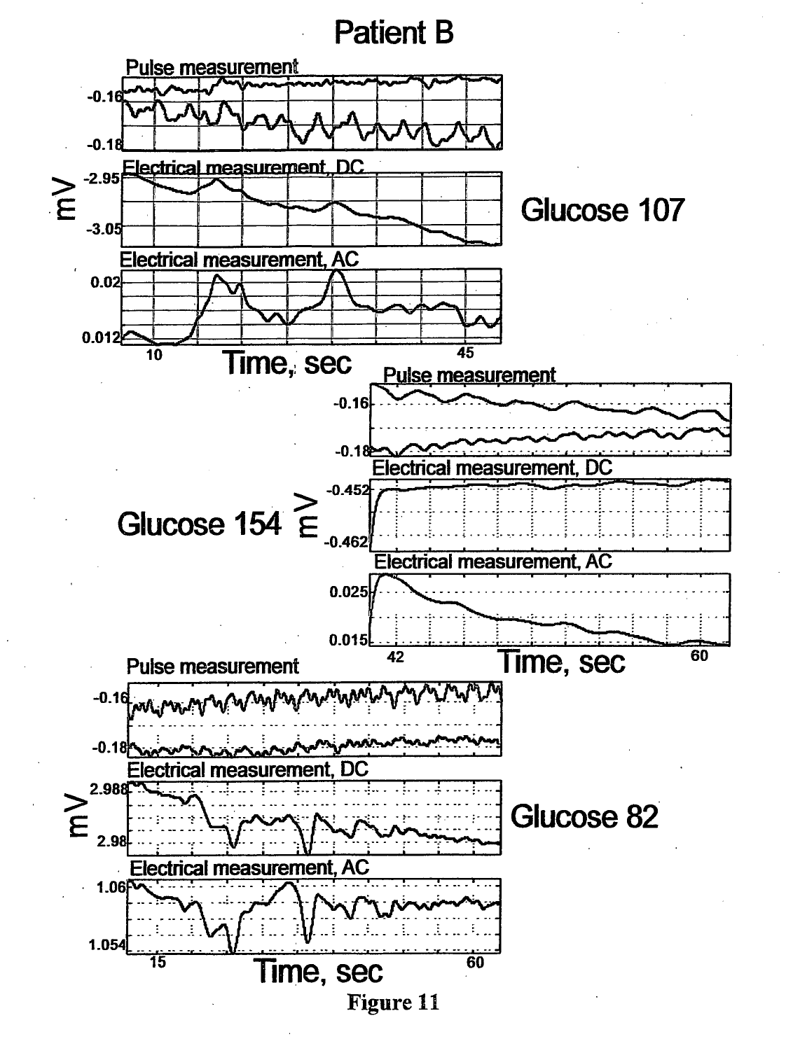

FIG. 11 is a graph showing experimental data generated by the present invention for a non-diabetic patient at three blood glucose levels;

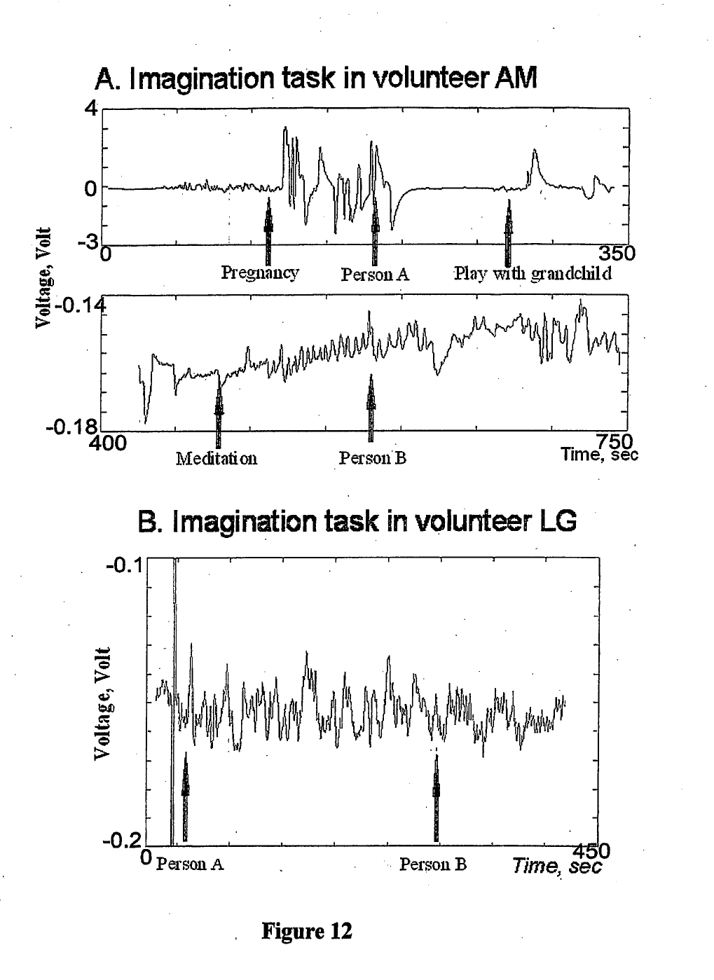

FIG. 12 is a graph showing experimental data generated by the present invention adapted to function as a lie detector;

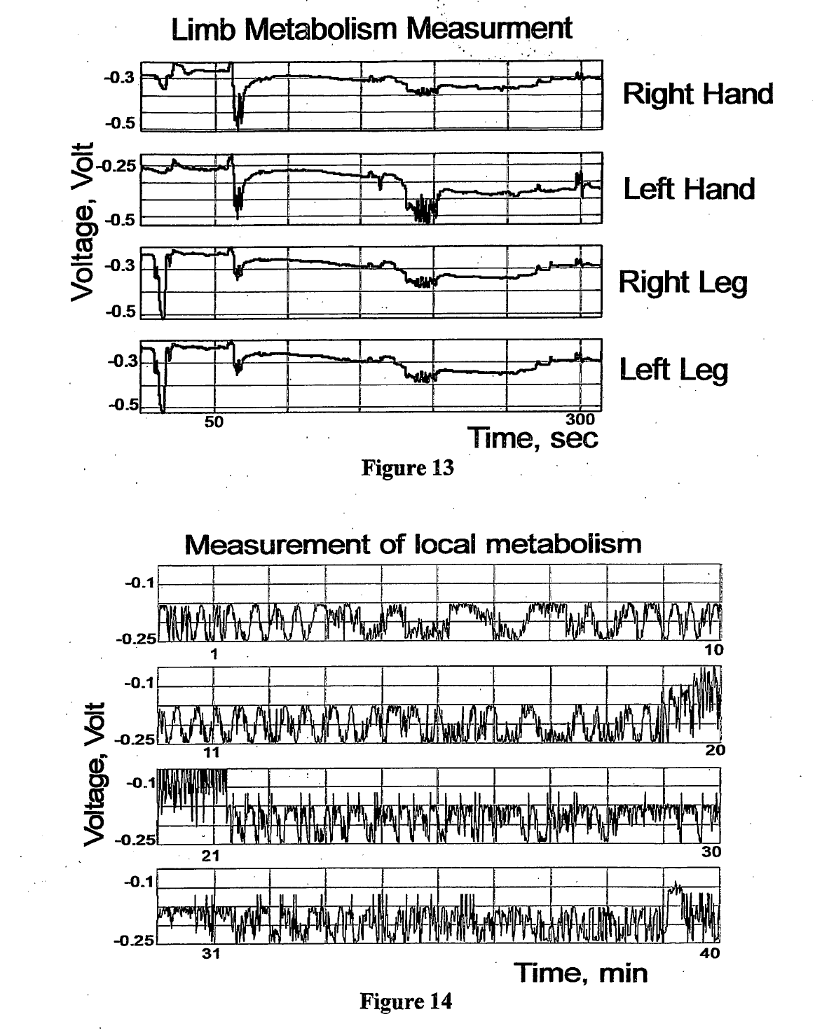

FIG. 13 is a graph of displaying experimental data generated by the present invention wherein the device is used to investigate limb metabolism;

FIG. 14 is a graph showing experimental data generated by the present invention for local metabolism disorder diagnostics;

FIG. 15 graphically shows experimental data generated by the present invention as a pharmaco-dynamics and pharmaco-kinetics tracking system; and

FIG. 16 is a top view of an exemplary embodiment of the device of the present invention constituted by an array of pads.

### DETAILED DESCRIPTION OF THE INVENTION

By way of example, the following description of the invention relates to its application as a glucose monitor. It should be understood that this is merely one application among an extensive list of applications of which the invention is capable.

Referring first to FIG. 1, there is shown a first embodiment of the present invention, adapted for glucose determination/monitoring, illustrated by a wrist watch or wristlet comprising three types of sensors: pulse-wave sensors 6a and 6b, biocompatible electrodes 7, and additional biocompatible electrodes 8a and 8b for detecting perspiration and estimating the acidity thereof.

The device comprises the following electronics: a keyboard 1, a body 2 with a display 3 and an electronic block 4. The keyboard 1 is supplied with a connector 5 to allow connection of a programmed cartridge, for example a home computer, cellular phone, palm-sized electronic notebook, etc (not shown). The body 2 incorporates the pulse-wave sensors 6a and 6b, biocompatible electrodes 7, and additional biocompatible electrodes 8a and 8b.

Electronic block 4 is supplied with an antenna 9 and a connector 10 for transferring data and/or an alarm signal through an external transmission-connection unit (not shown), (e.g. telephone line, fax, the Internet) for sending such data to a physician.

The device also includes two thermometers 11a and 11b for measuring the patient's skin and the surrounding temperature, respectively, and a 3-dimensional accelerometer 12 for measuring motion intensity or physical activity of the hand (not seen).

FIG. 2 is a block diagram of the components of the device including the operative connections between those components.

The following components are shown and labeled as indicated:
The two pulse-wave sensors 6a and 6b (PWS1 and PWS2), which are connected to a microprocessor (MP 6).
three electrodes 7 (El_1, El_2 and El_3), where electrodes El_1, El_2 are electrochemically connected to electrode El_3, which is a reference electrode (not seen in FIG. 1 as it is inside the electronic block 4). The three electrodes 7 (El_1, El_2 and El_3) are connected to three voltmeters V2, V3 and V4, respectively. In order to measure DC and AC voltages it is necessary to use the two separate voltmeters. Therefore the signal from the El_1 goes to V1 to measure acidity, to V2 to measure DC and to V3 to measure AC.
The two perspiration measuring electrodes 8a and 8b (AdEl_1 and AdEl_2), which are each connected with a voltmeter (V1, V2) [reference #'s?], respectively;
The 3-dimensional accelerometer 12 (Acc).
Two thermometers 11a and 11b (T-1 and T-2) for measuring skin and surrounding temperature, respectively.
Four microprocessors (MP1, MP2, MP3, MP4); and the programmed microprocessor MP6 connected to the keyboard 1; and a processor, MP5, with memory M connected thereto; and having a charge-connector unit and alarm system.

Note, the voltmeters and microprocessors referred to herein are not seen in FIG. 1 and so are not given reference numerals (merely labels as seen in FIG. 2), however, they are located within the electronic block 4.

The microprocessor MP1 is connected with PWS1 and it analyzes pulse-wave spectral characteristics using a standard mathematical software program package (e.g. Matlab or other software). The microprocessor MP2 is connected to PWS1, PWS2 and a timer/clock, and it measures a pulse wave propagation velocity and heart rate. The microprocessor MP4 is connected to PWS2 and it analyzes a pulse wave spectrum, for example using Matlab.

The above microprocessors MP1, MP2 and MP4 are connected with a programmed microprocessor MP5 having a display. The potential difference between electrodes 8a and 8b (AdEl-1 and AdEl-2) is proportional to the perspiration's acidity.

With reference to FIG. 3, the principles of pulse wave measurements use the following principles:
1. The rate of movement of the blood can be estimated by the rate of pulse wave propagation between sensors 6a and 6b.
2. The blood flow is proportional to the cross-section of arteries and the velocity of the blood.
3. Blood viscosity affects the shape of the pulse waves, the rate of their propagation and the pulse wave spectrum.

The following data are supplied to the programmed microprocessors from the various sensors:

1. Pulse wave area from PWS1,

2. Pulse wave spectrum from PWS1,

3. Pulse wave area from PWS2,

4. Pulse wave spectrum from PWS2,

5. Pulse wave propagation velocity,

6. Heart rate,

7. Indication of existence of perspiration, and

8. Acidity of perspiration.

For calibration purposes, the first data are compared in the programmed microprocessor MP5 with parameters (i.e. glucose level, blood pressure, heart rate, etc.) that were recorded in the processor's memory M during an oral glucose tolerance test (OGTT) and/or during an electrocardiogram (ECG) stress test. The results of such a calibration are input into an individual “mathematical model” resulting from an individual calibration with neural network software. Similar neural network software is used to estimate the following important parameters:

1. Blood glucose level,

2. Heart rate,

3. Blood flow,

4. Blood pressure,

5. Blood viscosity (which may be affected by dehydration).

The programmed microprocessor MP5 displays selected parameters on the display 3. It is connected with a processor P that can produce an alarm if selected parameters are beyond predetermined limits, which depend on the rate of change of the parameters.

The alarm (and parameters) may be transmitted through a cellular telephone or other means of communication. All of the parameters are periodically recorded in the memory M in case any deviations, for example, they may be transmitted daily into the computer of a physician, medical center, clinic, etc, through a separate charge-connection unit.

Preliminary examination of the other components of the device consisted of checking pulse-wave and bio-electricity diagnostics. The above-described theoretical basis of such diagnostics is explained with reference to FIGS. 4-6. Data for FIGS. 4 and 5 were generated from the Michaelis-Menten equation and the data for FIG. 6 were generated from the Lipman equation and electro-capillary curves.

The change of the rate of cellular glucose absorption as a function of the blood glucose level at a range of insulin levels (picomoles/ml) is shown in FIG. 4. The rate of glucose absorption depends on glucose and insulin blood level.

As seen, the maximal rate of glucose absorption is typically in a BGL range of 65 to 115 mg/dL, which corresponds to the maximal stability of the glucose level and more particularly to the maximal motion force and rate of return to equilibrium (as seen in FIG. 5.). The dominant parameter of any living system is metabolism, which includes in particular the equilibrium between carbohydrate metabolism and oxygen/carbon dioxide use and production.

FIG. 6 shows the function of Gibbs energy of healthy cells (indicated by diamond symbols) and cancer cells (indicated by square symbols). The relative Gibbs energy is relative to the average Gibbs energy of the cells; and the relative intensity of metabolism is relative to the 50% level of the normal basic metabolism value. Metabolism measurements, which are measurable using the device of the present invention, can provide estimation of cellular Gibbs energy and thus can provide important information in the treatment of cancer.

Thus Gibbs energy is dependent on the relative intensity of the metabolism. It shows that in the condition of both a metabolism that is too low or too high, the Gibbs energy of cancer cells is lower than that of healthy cells. Under this condition the rate of cancer cell division may be much higher than in healthy cells.

Furthermore, the separation between the curves in FIG. 6 shows that there is a Gibbs energy difference between cancer and healthy cells which allows the estimation of polymorphism of the cancer cells as the tendency for polymorphism is proportional to the difference in the Gibbs energy between the cancer cells and the healthy cells. Cancer polymorphism itself is a very important property of the cancer cells which directly affects treatment protocol decisions and the potential effectiveness of cancer treatment.

Experiments and measurements were made during oral glucose tolerance tests (OGTT), which included a blood glucose level measurement by a standard device “Accu-Chek” [Roche Diagnostics, Mannhelm, Germany].

In parallel, analysis of pulse waves and bio-potentials were performed using the device of the present invention. Pulse waves were measured by piezo-electric transducers and microphones in parallel with electrical signals during the measurements.

These signals produced from the above measurements were recorded in a computer by standard analog-to-digital protocol and were analyzed by standard mathematical programs (e.g. “MatLab”).

Pulse-wave measurements results obtained by the present device are shown in FIG. 7 (raw data), FIG. 8 (filtered data) and FIG. 9 (raw data after Fourier transform analysis). The characteristic forms of the recorded pulse waves using the pulse wave sensors 6a and 6b are shown in FIG. 7 at three blood glucose levels (130, 200 and 260 mg/dL).

Upon inspection of the curves of FIGS. 7-9, it is obvious that the form of the pulse wave and its spectral characteristics changes from BGLs of 130 to 260 mg/dL. For example, the downward sloping portions of the curves in FIG. 8 are much less smooth as the BGL increases. Therefore, such measurements can be correlated with BGL and thus BGL can be determined via those measurements consistent with the above-mentioned theory and by use of the device of the present invention.

With reference to FIG. 9, it can also be observed that as the BGL increases, there are more high frequency components (peaks P₁, P₂, P₃, P₄ and P₅). Again, such results can be used to form a correlation between the pulse-wave measurements and the BGL so that using the device of the present invention, BGL can be conveniently, continuously and non-invasively obtained.

In all the experiments described herein, wherein a DC voltage was recorded, a standard AgCl reference electrode was used as the reference electrode for the DC voltage measurements.

FIGS. 10 and 11 show results of simultaneously recorded pulse-wave and bio-potential measurements obtained by the present device (particularly by pulse-wave sensors 6a and 6b; and electrodes 7) and their processing at different BGLs, for a diabetic patient (patient A) and non-diabetic patient (patient B), respectively.

It can be seen from these graphs that with the change of the blood glucose level there is change in the spectral characteristics of the pulse waves and voltage measurements. Such change is a biological response of a patient to intolerant BGLs (i.e. above 120 mg/dL). The parameters of these characteristics analyzed by neural network algorithms allow transforming all these multi-parametric dynamical parametrical changes into blood glucose level estimation.

Thus, the two afore-mentioned experiments indicate that an at least semi-quantitative model can be achieved and used as the basis of the present invention, using measurements of the device of the present invention.

FIG. 12 shows the results of a further experiment involving two female volunteers (volunteer AM, aged 63 and volunteer LG, aged 56). The volunteers were connected to the device (particularly electrodes 7), in the supine position to avoid uncontrolled movement. During the measurements they were asked to recall different situations from life, including: (a) thinking about pregnancy, (b) thinking about another person, (c) meditation and (d) playing with grandchildren.

The time at which these thoughts were suggested are shown by arrows on the graphs of FIG. 12. It can be seen that typically after a brief delay of a few seconds, there is a clear change in the voltage characteristics. Such change shows that the voltage measurements (DC and low frequency AC together with high frequency AC) are capable of indicating a response to various psycho-emotional stimuli. Such measurements therefore have potential applications in lie detector machines and to psycho-immune measurements.

FIG. 13 shows the results of different voltage measurements, produced by the electrodes 7 of the device. A device was worn on each of all four limbs and corresponding DC and low and high frequency AC voltage changes were measured during heating of the left leg by an assistant (at about 65 seconds into the experiment); and later (at about 180 seconds into the experiment) with the volunteer heating his own hands using thought/imagination.

The perturbations seen in curves indicate that the device is capable of sensing metabolism and blood flow change in the limbs. Thus, the device can be used as a bio-feedback system and for diagnostics. Furthermore, the experimental results support a recently developed theory that there is a coordinated interconnection between the limbs. This in itself has an enormous importance for the diagnostics and treatment of limbs.

FIG. 14 is a graph showing experimental data generated by the present invention for a diagnostics of a local metabolism disorder. Here the device was worn on a portion of a 53 year old male patient having diseased skin with an affected metabolism. The graph shows dynamic voltage change during a bio-resonance electromagnetic treatment. For the first three minutes of the measurements, the patient was working by himself, i.e. using the device as a biofeedback system. At three minutes into the experiment, the patient fell asleep and an electromagnetic resonance treatment began wherein different resonance signals were used.

The change in voltage response seen in the curve of FIG. 14, at three minutes into the experiment when the resonance treatment began, validates the sensitivity of the electrode measurements to a change in local metabolism caused by the treatment. The device further monitored the patient's metabolism during continuation of the treatment, which was suspended temporarily between 28-31 minutes and after 39 minutes. Again, the electrodes measure changes in the patient's local metabolism as seen in the response change shown in FIG. 14 at those times.

FIG. 15 graphically shows experimental data generated by the present invention as a pharmaco-dynamics and pharmaco-kinetics tracking system. During this experiment a 64 year-old male volunteer, took a nutrient supplement and the electrodes 7 of the device were placed on his body at locations whereat the supplement was expected to act upon.

There is a clear affect in the dynamic voltage, in particular a 50 mV decrease, as a result of the supplement intake.

This indicates that the device can be used to track physiological changes in the body as a result of drug/supplement/food intake and thus it has application in pharmaco-dynamics, drug/supplement development, improvement of treatment protocols, diet programs and so on.

In FIG. 16 there is shown an embodiment of the device in which a pad 14 comprises an array of electrodes 8 (and/or sensors 6, or combination thereof) arranged on it. In such an arrangement, voltage measurements can be made between electrodes 8 and such a pad 14 can be conveniently disposed at virtually any location on the surface of a biological being. The pad 14 is convenient for use in performing organ metabolic measurements, for example.

For clarity, a summary of the particular electrodes/sensors/meters required for different embodiments of the device of the present invention is shown in the table below.

Required Sensors for Particular Embodiments of the Device

| | Sensors | | | | |
|---|---|---|---|---|---|
| | Basic | | | | |
| | Building | Pulse wave | Acoustic | | |
| Device | Block** | sensors | sensors | Thermosensor | Accelerometer |
| Glucose monitor | 1 | No* | No* | 1 | 1 |
| Nervous system | 1 | No* | No* | No* | No* |
| monitor | | | | | |
| Wireless | 5 | No* | No* | No* | No* |
| ECG | | | | | |
| Local metabolism | At least 2 | 2 | No* | At least 2 | No* |
| monitor | | | | | |
| Limb metabolism | 4 | 8 | 4 | 4 | 4 |
| monitor | | | | | |
| Psychological | At least 4 | 8 | 4 | 4 | No* |
| detector, | | | | | |
| Lie detector | | | | | |
| Pharmacokinetic; | At least 4 | 8 | At least 4 | At least 4 | At least 4 |
| pharmacodynamic | | | | | |

No* = not required in the most simplistic embodiments of the device, however could be required in more complex embodiments.
** = two spaced apart electrodes at least one of which is in contact with the biological being for providing a bio-potential measurement including a low frequency AC voltage and/or a DC voltage in which one of the two electrodes is a reference electrode providing a reference for the DC voltage.

It is important to mention that the implementation of the device being a BB as an ECG provides a compact, user friendly wireless ECG device. The fact that measurements are accomplished by an electrode with reference to a reference electrode allows voltage measurement without connecting an electrical loop through the biological being itself.

Thus present device and method allows monitoring of a patient's physiological (health/illness) condition by measurement, recording and analysis of the patient's functional physiological profile.

It is very important to note that some of the above-mentioned parameters can be measured using merely DC voltage and/or low frequency AC voltage and do not necessarily need both.

1. A device for measuring physiological parameters of a biological being comprising: at least two spaced apart electrodes at least one of which is in contact with the biological being for providing a bio-potential measurement including a low frequency AC voltage and/or a DC voltage in which one of said at least two electrodes is a reference electrode providing a reference for said DC voltage, wherein said low frequency AC voltage and/or DC voltage of said bio-potential measurement is used to determine said physiological parameters.

2. The device according to claim 1, wherein the electrodes further measure high frequency AC voltage.

3. The device according to claim 1, wherein the physiological parameters include at least one of blood glucose level, cardiovascular function, blood pressure, organ function, tissue function, brain function, neural function, local and/or limb metabolic condition, pharmacokinetics, pharmaco-dynamics psychological condition, temperature, or any combination of these and their trends.

4. The device according to claim 1, wherein it further comprises additional contact or non-contact sensors of any one, or combination of the sensor types including: pulse-wave, motion, temperature, acoustic, electro-magnetic, acidity and perspiration.

5. The device according to claim 1, wherein at least some of the electrodes are used in passive sensors.

6. The device according to claim 1, wherein the electrodes are two or more biocompatible electrodes composed of different conductive materials.

7. The device according to claim 1, wherein it is multi-parametric and multi-diagnostic device.

8. The device according to claim 1, wherein at least two of the electrodes are disposed in spaced apart cross-sections of a limb of the biological being.

9. The device according to claim 1, wherein all its sensors and electrodes are of the non-invasive type.

10. The device according to claim 1, wherein it is adapted to be a continuous measuring device thereby allowing monitoring of the physiological parameters.

11. The device according to claim 1, wherein it is adapted to provide trend analysis of the physiological parameters.

12. The device according to claim 1, wherein the electrodes are constituted by an array of electrodes and/or sensors or combination thereof.

13. The device according to claim 12, wherein measurements can be made between any of the electrodes or sensors of the array.

14. The device according to claim 1, wherein it is adapted to be worn on a limb, head or torso of the biological being.

15. The device according to claim 1, further comprising a signal processor for providing a warning of critical changes and/or rate of changes in the physiological parameters of the biological being.

16. The device according to claim 1, further comprising a programmed cartridge for individual periodic calibration of the device during routine clinical calibration tests.

17. The device according to claim 1, further comprising a data input unit for inputting any one of (a) the results of blood glucose measurements obtained by known methods to serve as reference values for calibrating said device and providing an indication of changes occurring in the patient's condition, (b) dosages of the patient's insulin, (c) oral hypoglycemic drugs, (d) patient's food intake and (e) medications used by the biological being.

18. The device according to claim 1, further comprising, or adapted for use with, a display for displaying one or more of (a) data input to the device, (b) measurements made by the device and (c) parameters calculated by the device.

19. A method for measuring physiological parameters of a biological being comprising: (a) providing a device according to claim 1; (b) contacting said device with a biological being, (c) measuring at least a DC voltage and/or a low frequency AC voltage of the biological being.

20. The method according to claim 19, further comprising obtaining baseline physiological data of a patient and comparing the measured physiological parameters of the biological being with said baseline data.

21. A device for use in measuring one or more physiological parameters of a biological being, the device being configured as a passive sensing device and comprising at least two spaced apart electrodes, at least one said at least two electrodes being intended to be in contact with the biological being for providing a bio-potential measurement including a low frequency AC voltage and/or a DC voltage, one of said at least two electrodes being a reference electrode providing a reference for the DC voltage, the device thereby enabling use of the low and high frequency AC voltage and/or DC voltage of the bio-potential measurement to determine one or more physiological parameters.

22. A device for use in measuring one or more physiological parameters of a biological being, the device being configured as a passive sensing device and comprising: biocompatible electrodes including at least two electrodes composed of different conductive materials.
